(19) United States
(12) Patent Application Publication
(10) Pub. No.: US 2014/0213909 A1
(43) Pub. Date: Jul. 31, 2014

(54) CONTROL-BASED INVERSION FOR ESTIMATING A BIOLOGICAL PARAMETER VECTOR FOR A BIOPHYSICS MODEL FROM DIFFUSED REFLECTANCE DATA

(71) Applicant: XEROX CORPORATION, Norwalk, CT (US)

(72) Inventors: Lalit Keshav MESTHA, Fairport, NY (US); Palghat Srinivas RAMESH, Pittsford, NY (US); Alvaro Enrique GIL, Rochester, NY (US)

(73) Assignee: XEROX CORPORATION, Norwalk, CT (US)

(21) Appl. No.: 13/755,155

(22) Filed: Jan. 31, 2013

(51) Int. Cl. A61B 5/00; A61B 5/1455; A61B 5/0205; A61B 5/107

(52) U.S. Cl. CPC A61B 5/0077; A61B 5/1075; A61B 5/14551; A61B 5/444; A61B 5/443; A61B 5/0205; A61B 5/7246; A61B 5/742; A61B 5/7475; A61B 5/0075
USPC 600/476

(57) ABSTRACT

What is disclosed is a system and method for estimating a biological parameter vector for a biophysics model using reflectance measurements obtained from a reflectance-based spectral measurement system. The present method uses a semi-empirical biophysics model to describe skin properties and estimate reflectance spectra and reduces the dimensionality of the estimated and measured reflectance spectra using basis vectors for computational efficiency. A mixture of algorithms are employed to generate an initial set of parameters which, in turn, are further refined using an iterative control based technique in which the error between the parameters derived from the measured spectra are compared to the parameters calculated from the estimated spectra. These errors are then processed to generate a small delta to the initial set of parameters. The process is repeated until an error between the estimated virtual biological parameters and the measured virtual biological parameters falls to zero or is otherwise below a pre-defined threshold level.

### TECHNICAL FIELD

The present invention is directed to systems and methods for estimating a biological parameter vector for a biophysics model from spectrum measurements obtained using a reflectance-based spectral measurement system taken in-vivo of the surface of an area of exposed skin.

### BACKGROUND

Skin cancers are an increasing problem around the world and account for about 40% of all diagnosed cancers in humans. Most skin cancers are curable, if detected sufficiently early enough. Currently, clinical dermatologists rely on visual inspection and personal experience to make an initial assessment of a lesion seen on the skin surface. Suspicious lesions are biopsied for analysis. Biopsy often involves the removal of some or all of the skin wherein the lesion resides with the extracted tissue being sent to a laboratory for analysis. Biopsy can be an unpleasant experience for most patients because the dermis and hypodermis layers are composed of cells and connective tissues which are perfused with blood vessels and flush with nerves. Dermatologists would greatly benefit from a non-invasive technique that could assist them in their clinical diagnostic decisions without having to physically remove skin tissue from the patient.

Approximately half of the blood volume in the dermis layer is occupied by red blood cells which transport oxygen. Oxygen is carried in hemoglobin molecules. In addition to knowing the blood volume fraction in the tissue, oxygen saturation can provide a good indication of hemodynamic activity within the tissue and is further a good indicator of tissue health. Oxygen saturation, as measured by the pulse oximetry, provides a global indicator of the clinical state of the patient but lacks from obtaining the oxygenation in-vivo localized to a particular region of the tissue in the dermis layer. In-vivo measurements of the thickness of an epidermal layer, melanin and blood concentration in human skin are considered useful for medical and cosmetic applications because skin color is mainly determined by the amount of melanin in the epidermis layer and blood volume fraction in the dermis layer. Prior art methods such as, for example, optical coherent tomography can acquire measurements of various skin parameters but are subject to noise from scattering and sound effects which may limit accuracy. In addition, the optical properties of the skin tissue layers implies that the light is scattered strongly and anisotropically throughout the visible spectrum. This makes simple models such as Beer's law poor approximations of skin optics. Monitoring of blood volume and tissue oxygenation as part of hemodynamic analysis can be performed non-invasively using diffused reflectance measurements provided the inversion can be performed accurately. This art would benefit greatly from a fast and accurate inversion method.

Accordingly, what is needed in this art is sophisticated control based inversion technique which uses diffused reflectance data obtained in-vivo from an unobstructed surface of the skin for accurate estimation of skin properties such as, skin thickness, melanin concentration, dermal blood volume, oxygen saturation, and the like, in a non-invasive, non-contact, remote sensing environment.

### INCORPORATED REFERENCES

The following U.S. patents, U.S. patent applications, and Publications are incorporated herein in their entirety by reference.
“Retrieving Skin Properties From In Vivo Spectral Reflectance Measurements”, D. Yudovsky and Laurent Pilon, Journal of Biophotonics Vol. 4, No. 5, pp. 305-314, (2011).
“Estimation Of Optical Properties Of Normal And Diseased Tissue Based On Diffuse Reflectance Spectral Model”, Shanthi Prince and S. Malarvizhi, Proceedings of the World Congress on Engineering, Vol. 1, WCE 2010, Jun. 30-Jul. 2, 2010, London, U.K. ISSN: 2078-0958.
“Rapid And Accurate Estimation Of Blood Saturation, Melanin Content, And Epidermis Thickness From Spectral Diffuse Reflectance”, D. Yudovsky and Laurent Pilon, Applied Optics, Vol. 49, No. 10, (April 2010).
“Simple And Accurate Expressions For Diffuse Reflectance Of A Semi-Infinite And Two-Layer Absorbing And Scattering Media”, D. Yudovsky and Laurent Pilon, Applied Optics, Vol. 48, No. 35, pp. 6670-6683, (December 2009).
“Modeling Diffuse Reflectance From Homogeneous Semi-Infinite Turbid Media For Biological Tissue Applications: A Monte Carlo Study”, George Zonios and Aikaterini Dimou, Biomedical Optics Express, Vol. 2, No. 12, pp. 3284-3294, Optical Society of America (2011).
“Modeling Diffuse Reflectance From Semi-Infinite Turbid Media: Application To The Study Of Skin Optical Properties”, George Zonios and Aikaterini Dimou, Biomedical Optics Express, Vol. 14, No. 19, pp. 8661-8674, Optical Society of America (2006).
“The Reflectance Spectrum Of Human Skin”, Elli Angelopoulou, Dept. of Computer and Information Science, University of Pennsylvania, GRASP Laboratory, Technical Report MS-CIS-99-29, (December 1999).
“Practical Genetic Algorithms”, Randy L. Haupt and Sue Ellen Haupt, Wiley-Interscience, 2^{nd} Ed. (2004), ISBN-13: 978-0471455653.
“Human Anatomy and Physiology”, Elaine Nicpon Marieb, Benjamin-Cummings Publishing; 9^{th} Ed. (2012), ISBN-13: 978-0321696397.
“Principles of Anatomy and Physiology”, Gerard J. Tortora and Bryan H. Derrickson, Wiley; 13^{th} Ed. (2011), ISBN-13: 978-0470565100.
“Control of Color Imaging Systems: Analysis and Design”, Lalit K. Mestha and Sohail A. Dianat, CRC Press (2009), ISBN-13: 9780849337468.

### BRIEF SUMMARY

What is disclosed is a system and method for estimating a biological parameter vector (a vector of biological parameters) for a biophysics model from measured spectrum obtained from a reflectance-based spectral measurement system. The present method uses a semi-empirical biophysics model to describe skin properties and estimate reflectance spectra. A mixture of algorithms are employed to generate an initial set of biological parameters (a vector) which, in turn, are further refined using an iterative control-based technique in which the norm of the error vector between these biological parameters derived from the measured spectra are compared to the biological parameters calculated from the estimated spectra. The errors are processed to generate a small delta to the initial set of biological parameters. The process is repeated until the error between the estimated virtual biological parameters and the measured virtual biological parameters falls to zero or is otherwise below a pre-defined threshold level. The teachings hereof enable the generation of an accurate biological parameter vector quickly. The present method reduces the dimensionality of the estimated and measured spectra using natural basis for the dimensionality reduction for computational efficiency. The natural basis enables the selection of a smaller set of spectral bands. The biological parameter vector obtained hereby effectuates improved accuracy in estimating various skin properties such as, skin thickness, melanin concentration, dermal blood volume, oxygen saturation, and the like, from measured reflectance spectra obtained in-vivo from the surface of the patient's skin.

One embodiment of the present method for estimating a biological parameter vector for a biophysics model from reflectance measurements obtained from a reflectance-based spectral measurement system involves performing the following. Measured spectrum Rₘ(λ) are received comprising in-vivo spectral reflectance measurements obtained using a spectral reflectance sensing device at wavelength λ from the skin surface. That surface is represented, at least in part, by a biophysics model for which a biological parameter vector P is desired to be estimated. The biophysics model uses an estimated virtual biological parameter vector to generate values of estimated spectrum Rₑ(λ). In various embodiments, the biophysics model comprises a model of multi-layered skin tissue and the biological parameter vector comprising, for example, epidermal thickness, melanin concentration, dermal blood volume fraction, skin oxygen saturation, and a light scattering parameter. The measured spectrum are transformed to a low dimensional virtual parameter space represented by a measured virtual biological parameter vector Pₘ. On a first iteration hereof, an initial biological parameter vector P₀ is provided to the biophysics model to obtain estimated spectrum which, in turn, are transformed to a low dimensional virtual parameter space represented by an estimated virtual biological parameter vector Pₑ. The following steps (A)-(B) are then iteratively performed until the norm of the error vector is at or below an acceptable threshold level. In step (A), the measured virtual biological parameter vector Pₘ is compared to the estimated virtual biological parameter vector Pₑ to determine an error E therebetween. In step (B), if the norm of the error vector is less than a pre-defined threshold value, the last estimated virtual biological parameter vector is the desired final estimated virtual biological parameter vector P_{F}. Otherwise, a next biological parameter vector is generated based upon the determined amount of error. This next biological parameter vector is provided to the biophysics model to obtain a next estimated spectrum. The next estimated spectrum is then transformed to a low dimensional virtual parameter space represented by a next estimated virtual biological parameter vector. This next estimated virtual biological parameter vector is used on the next iteration. Steps (A)-(B) are repeated until the norm of the error vector is determined to be within an acceptable limit. Thereafter, the final estimated virtual biological parameter vector P_{F} is communicated to a memory or storage device. Various embodiments are disclosed herein in greater detail.

Many features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 illustrates the basic structures of human skin;

FIG. 2 shows a pair of human hands with the left hand having a single lesion (at 201) thereon and the right hand having two lesions (at 202 and 203) on the skin;

FIG. 3 shows a hyperspectral camera available from IMEC which is a fully integrated CMOS compatible hyperspectral sensor consisting of a set of spectral filters that are directly post-processed at wafer level on top of a commercially available CMOSIS CMV4000 image sensor;

FIG. 4 illustrates one embodiment of an example reflectance-based spectral measurement system for capturing reflectance measurements from the surface of a human hand for estimating a biological parameter vector in accordance herewith;

FIG. 5 shows a comparison of the K-M model to the Monte Carlo model for diffuse reflectance data from a semi-infinite media with a refractive index of n=1;

FIG. 6 shows a comparison of the semi-empirical K-M model with Monte Carlo;

FIG. 7 shows a semi-empirical K-M model derived for a two layer geometry comprised of a thin (finite) top layer and a semi-infinite bottom layer;

FIG. 8 shows a comparison of the semi-empirical K-M to Monte Carlo for the two layer structure of FIG. 7;

FIG. 9 shows one embodiment of a block diagram of a system for the control-based inversion of the two-layer skin model;

FIG. 10 is a flow diagram illustrating one embodiment of the present method for estimating a biological parameter vector for a biophysics model;

FIG. 11 is a continuation of the flow diagram of FIG. 10 with flow processing continuing with respect to either node A or node B;

FIG. 12 plots various test spectra and performance results of an implementation of the control-based inversion method disclosed herein;

FIG. 13A-B show a table of a range of skin parameters (13A) and a table of parameters used by the Genetic Algorithm (13B);

FIG. 14 is a table showing for estimated virtual biological parameter values generated using the teachings disclosed herein, as compared to actual values;

FIG. 15 illustrates a block diagram of one example image processing system for implementing various aspects of the present method as shown and described with respect to the flow diagrams of FIGS. 10 and 11; and

FIG. 16 illustrates a block diagram of one example special purpose computer for implementing one or more aspects of the present method as described with respect to the flow diagram of FIGS. 10 and 11, and the various modules and processing units of the image processing system of FIG. 15.

### DETAILED DESCRIPTION

What is disclosed is a system and method for estimating a biological parameter vector for a biophysics model from reflectance measurements using a reflectance-based spectral measurement system. The objective hereof is to produce an estimated virtual biological parameter vector from measured spectrum. A semi-empirical biophysics model is employed which describes the biological variability of skin. Methods are utilized to reduce the dimensionality of the estimated and measured reflectance spectra during each measurement. Dimensionality reduction effectively enables one to operate in a virtual parameter space wherein the data can be more readily manipulated.

It should be understood that one of ordinary skill in this art would be readily familiar with acquiring spectral reflectance measurements using a spectral reflectance sensing device and for manipulating spectral data. One skilled in this art would have a working understanding of Simultaneous Perturbation Stochastic Approximation, the Levenberg-Marquardt Algorithm, and Genetic Algorithms. Additionally, one of ordinary skill would also be familiar with techniques for converting high dimensional data to a low dimensional virtual parameter space, including classical and Bayesian approaches to linear and nonlinear problems and multi-criteria optimization methods and algorithms.

### NON-LIMITING DEFINITIONS

A “biological entity” refers to any subject of interest having a region of exposed skin from which measured spectrum can be obtained and processed in accordance with the teachings disclosed herein. Although the term “human”, “person”, or “patient” may be used at various points throughout this disclosure, it should be appreciated that a biological entity to which the present invention is directed may be something other than a human. As such, the use of “person”, “patient” or “human” is not to be viewed as limiting the scope of the appended claims strictly to human beings.

A “region of exposed skin” refers to an unobstructed area of a surface of skin from which spectral reflectance measurements can be obtained.

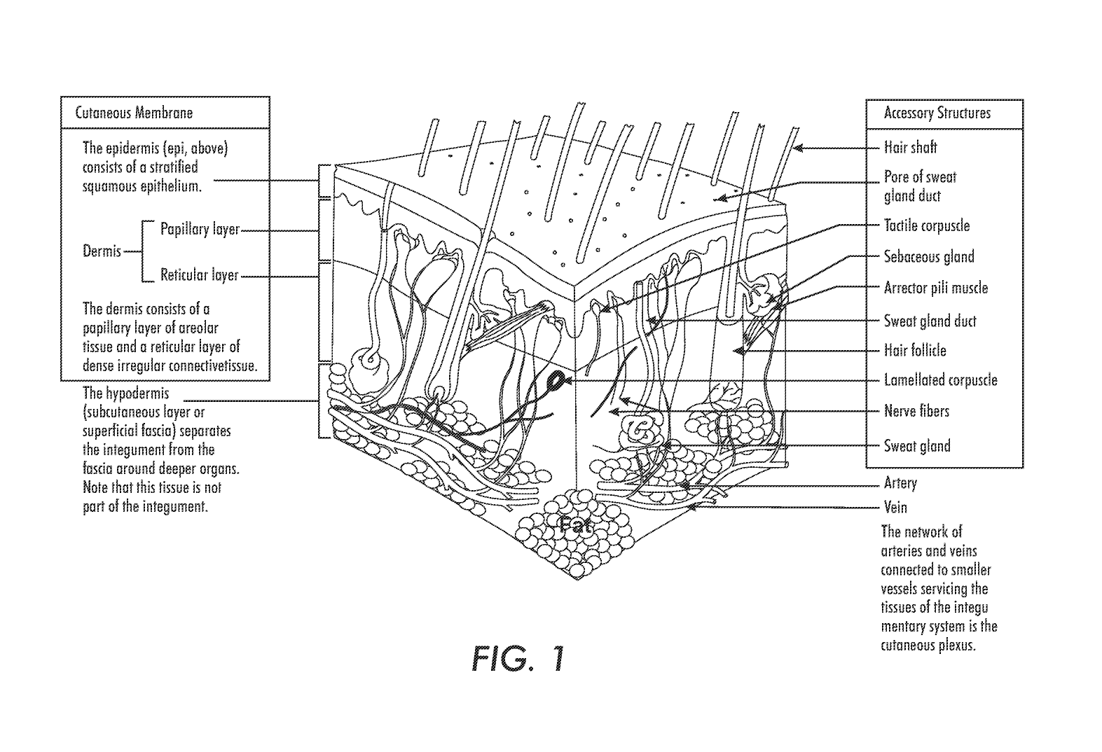

“Skin” protects underlying tissues, internal organs, and other anatomical structures against impact, abrasion, ultraviolet radiation, chemical exposure, to name a few. FIG. 1 shows a cross-section of human skin illustrating the basic structures thereof. Skin accounts for approximately 16% of total body weight. Skin is flush with nerves which provide the brain with sensory data regarding physical contact with the outside world. As shown in FIG. 1, skin comprises three layers, i.e., epidermis, dermis and hypodermis layers. The epidermis is bloodless and dominated by epithelial cells and relies on diffusion of nutrients and oxygen from capillaries within the dermis layer. The primary pigments involved in skin coloration are carotene and melanin. Both pigments are present in the epidermis. Melanocytes in the epidermal layer produce various shades of pigment called melanin which protect underlying tissues from ultraviolet radiation. The dermis layer lies between the epidermis and hypodermis layers and consists of multiple layers with networks of blood vessels, lymphatic structures, nerve fibers and accessory organs such as hair follicles and sweat glands. The hypodermis layer is dominated by adipose (fat) tissue. The hypodermis layer serves as a boundary between skin structures and the rest of the body.

“Skin cancer” refers to a growth or lesion on the skin which is cancerous. Most skin cancers arise in the outer (epidermis) layer although some cancers appear within the deeper structures. There are three common skin cancers, i.e., basal cell carcinoma, squamous cell carcinoma, and melanoma. Generally, any growth (tumor) or abnormal discoloration (lesion) on the skin that increases in size over time is suspicious of being a skin cancer. Embodiments hereof are particularly directed to the facilitation of skin cancer detection and diagnosis.

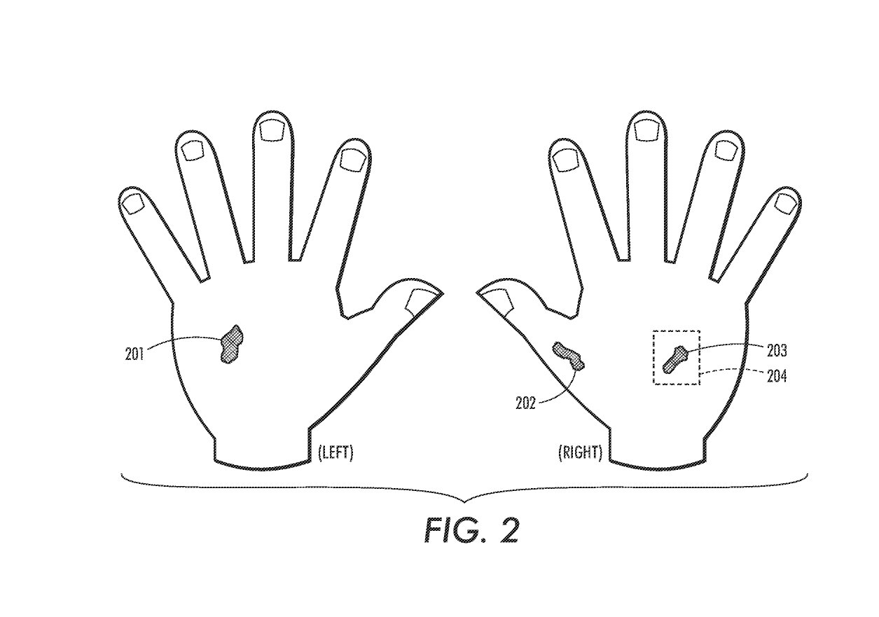

A “region of interest” is an area of exposed skin. FIG. 2 shows a pair of illustrative hands with the left hand having a single mark 201 and with the right hand having two marks 202 and 203. If those implementations wherein the teachings hereof are used for skin cancer diagnosis, the mark itself is of interest. A region of interest may be an area around the mark of interest. One such region is shown at 204 around mark 203. A lesion on the skin can be segmented to determine a boundary separating the lesion from surrounding normal skin thus restricting the computational complexity hereof to only those pixels within that region taken from sequences of images captured at different wavelengths using a spectral reflectance sensing device.

A “spectral reflectance sensing device” is an imaging system with spectral image capturing capability. Such an imaging system produces spectral measurement acquired for each pixel in an image. A spectral reflectance sensing device can be a spectrometer, a spectrophotometer, a multi-spectral camera, and a hyperspectral camera, as are readily known in the arts. In another embodiment, the spectral reflectance sensing device is a hybrid imaging system capable of capturing both color and spectral data. A spectrophotometer is a photometer that can measure intensity as a function of the light source wavelength. Important features of spectrophotometers are spectral bandwidth and linear range of absorption or reflectance measurement. Spectrophotometers only provide spot measurements. A spectrometer is an optical instrument which separates optical signals according to their wavelengths. These specialized instruments come with different spectral responses and are available from vendors in various streams of commerce. Spectrometers can be customized with probes and different light sources (e.g., tungsten halogen light) to measure reflected light from surfaces.

A “multi-spectral camera” can be either a multi-spectral or a hyper-spectral imaging system. Both embodiments generally comprise an array of spectral sensors which measure light reflected from a target. A multi-spectral camera can operate in the visible wavelength band or in the IR wavelength band or in both bands. A multi-spectral camera typically has at least one light source for illuminating the object and a detector array with each detector having a respective narrow band-pass filter. In different embodiments, a multi-spectral camera includes a plurality of outputs for outputting reflectance values on a per-channel basis, and may further comprise a processor and a storage device for processing and storing reflectance values. Such a camera system also may incorporate a storage device, a memory, and a processor capable of executing machine readable program instructions.

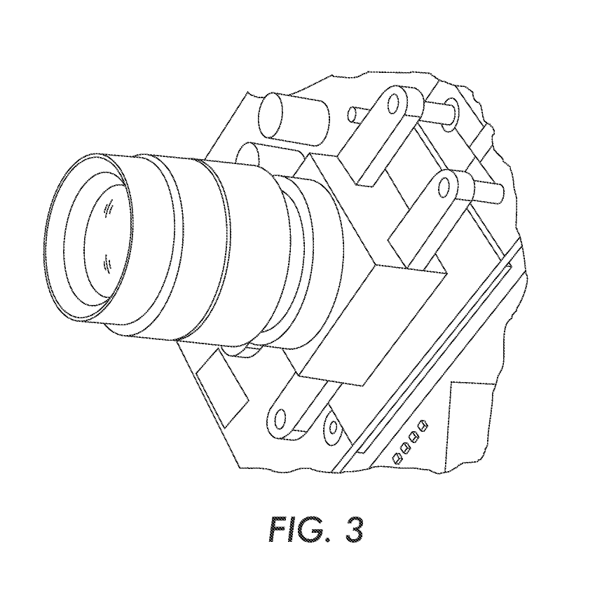

A “hyperspectral camera” combines spectroscopy and imaging and thus can discriminate between different objects that cannot be accurately distinguished using traditional RGB imaging methods. Most hyperspectral cameras owe their spectroscopic ability to a diffraction grating which spreads the light from a narrow slit-shaped aperture over a sensor. If the slit is oriented in the x direction, then sweeping the aperture over a scene by means of a movable mirror builds the image in the y direction. The narrow slit and long focal length yield fine spectral and spatial resolution, but at the expense of throughput (because the aperture is small), camera size (because of multiple optical components), and mechanical complexity (because the optics are moveable). On such hyperspectral camera, as shown in FIG. 3 (by the IMEC Corporation of Belgium), is a fully integrated CMOS compatible hyperspectral sensor.

“Measured spectrum”, denoted Rₘ(λ), refers to reflectance measurements obtained using a spectral reflectance sensing device at wavelength λ.

“Receiving measured spectrum” is intended to be widely construed and means to retrieve, receive, capture, download, or otherwise obtain spectral measurements for processing in accordance with the methods disclosed herein. Values for measured spectrum may be received as individual values, or received as a continuous stream of spectral data in real-time. Measured spectrum may be received on a continuous basis from the spectral reflectance sensing device or retrieved from a remote device over a wired or wireless network. In other embodiments, the measured spectrum are processed, in whole or in part, by one or more processors within the spectral sensing device, with a result thereof being provided by the device as output.

A “biological parameter vector”, generally denoted as P, refers to a vector of biological parameters. In those embodiments where the systems and methods hereof are used for analysis of skin, the biological parameters would be any of: epidermal thickness, melanin concentration, dermal blood volume fraction, skin oxygen saturation, and a light scattering parameter.

An “initial biological parameter vector”, denoted P₀, is a biological parameter vector which is provided, on a first iteration, to the biophysics model to obtain estimated spectrum. An initial biological parameter vector is generated using, for example, a Simultaneous Perturbation Stochastic Approximation (SPSA), a Levenberg-Marquard Algorithm (LMA), or a Genetic Algorithm, as are widely understood. Briefly, the SPSA is a descent method for finding global minima. Its main feature is the gradient approximation that requires only two measurements of an objective function, regardless of the dimension of the underlying optimization problem. As an optimization technique, it is well suited to adaptive modeling and simulation and is widely used for optimizing systems with multiple unknown parameters. Examples are provided at the SPSA website. The Levenberg-Marquardt Algorithm (LMA) provides a numerical solution to the problem of minimizing a function, generally nonlinear, over a space of parameters of the function. These minimization problems arise especially in least squares curve fitting and nonlinear programming. Essentially, LMA interpolates between the Gauss-Newton Algorithm (GNA) and the method of gradient descent. LMA is typically more robust than GNA which means that, in many cases, it can find a solution even when it starts far off the final minimum. LMA is a popular algorithm used in many software applications for solving generic curve-fitting problems. However, LMA finds only a local minimum, not a global minimum. A Genetic Algorithm (GA) is a search heuristic that mimics the process of natural evolution. GA belongs to a larger class of Evolutionary Algorithms (EA) used to generate solutions to optimization and search problems. The reader is respectfully directed to the above-incorporated text entitled: “Practical Genetic Algorithms”, Wiley-Interscience, 2^{nd} Ed. (2004), ISBN-13: 978-0471455653.

A “semi-empirical biophysics model” or simply “biophysics model”, is a model which receives, as input, a vector of biological parameters and which generates, as output, estimated spectrum.

“Estimated spectrum”, denoted Rₑ(λ), refers to spectrum which are estimated (as opposed to the measured spectrum) and are produced by the biophysics model. In one embodiment, the estimated spectrum is defined by the following relationship:

$${\underline{R}}_{e} ( λ ) = {\sum }_{i = 1}^{N} {P}_{i} {\underline{Ψ}}_{i} ( λ ) \qquad ( 1 )$$

where Pᵢ is the i^{th} parameter of biological parameter vector P, ψᵢ(λ) is the i^{th} column-wise basis vector with each element along a given row representing a basis value for wavelength λ, and N is the number of parameters. The basis set is constructed by Design of Experiments (DOE) on the biophysics model or by Monte Carlo simulation.

A “measured virtual biological parameter vector” is a vector of biological parameters obtained by having transformed the received measured spectrum Rₘ(λ) to a low dimensional virtual parameter space represented by vector Pₘ. In one embodiment, vector Pₘ is defined by the following relationship:

Pₘ=[ψ^{T}(λ)ψ(λ)]⁻¹ψ^{T}(λ)Rₘ(λ) (2)

where ψ(λ) is a column-wise basis vector with each element along a row representing a basis value for wavelength λ, and T is a transpose operation.

An “estimated virtual biological parameter vector” is a result of having transformed the estimated spectrum Rₑ(λ) to a low dimensional virtual parameter space represented by vector Pₑ. In one embodiment, vector Pₑ is defined by the following relationship:

Pₑ=[ψ^{T}(λ)ψ(λ)]⁻¹ψ^{T}(λ)Rₑ(λ) (3)

where ψ(λ) is a column-wise basis vector with each element along a row representing a basis value for wavelength λ, and T is a transpose operation.

A “next estimated virtual biological parameter vector” is a vector of estimated virtual biological parameters obtained for use on a next iteration. As more fully disclosed herein, the next estimated virtual biological parameter vector is determined using a feedback controller comprising a MIMO integral controller with a gain matrix K where the gain matrix is designed using either a pole-placement strategy, or a Linear Quadratic Regulator (LQR) by having computed a Jacobian matrix at nominal parameter values.

A “final estimated virtual biological parameter vector”, denoted P_{F}, refers to a last estimated virtual biological parameter vector output by the iterative process when the error determined as a result of a comparison between the measured virtual biological parameter vector and the next estimated virtual biological parameter vector is at or below a threshold level.

A “storage device” refers to a device where a digital representation of a result can be stored. Results include, for instance, numbers, parameters, text, formulae, and the like. Storage devices are well known in the arts and include RAM, ROM, CD-ROM, DVD, flash drives, hard drives, floppy disk, and other media capable of storing data.

A “remote sensing environment” refers to the non-contact, unobtrusive, non-invasive acquisition of spectral measurements such that the resting patient remains undisturbed during data acquisition.

### Example Spectral Reflectance Measurement System

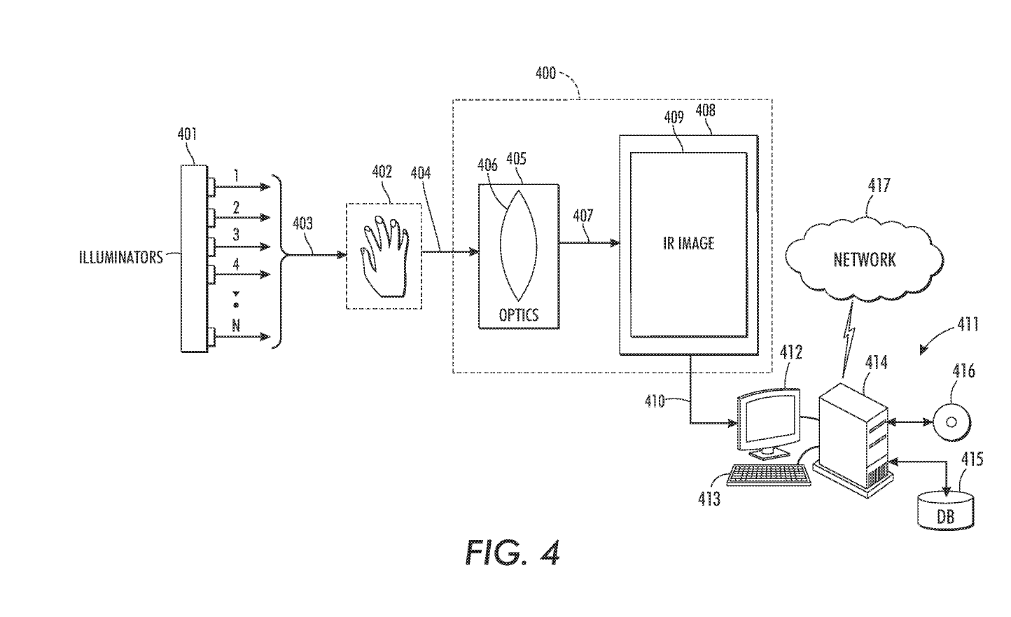

Reference is now being made to FIG. 4 which illustrates one embodiment of an example reflectance-based spectral measurement system 400 for capturing measured spectrum from the surface of a human hand for estimating a biological parameter vector for a biophysics model in accordance with the teachings hereof.

In FIG. 4, example human hand 402 reflects a light beam, collectively at 403, emitted at any of a variety of wavelengths by illuminators 401 such that at least a portion of the reflected light 404 is received by optics 405 of the spectral measurement system 400. Optics 405 has one or more lens 406 which serves to focus the received reflected light 404. Such optics may include one or more band pass filters that only allow light in a narrow band of a desired wavelength to pass through. Filters may be sequentially changed to acquire N wavelength bands of the same image. Focused light 407 is directed onto an array of detectors 408 which independently record intensity values at multiple pixel locations along a multi-dimensional grid such that the received light is spatially resolved to form an IR image 409. In one embodiment, detector array 408 comprises a multi-spectral IR detection device whose spectral content is selectable. Suitable optics 405 and detector array 408 are commonly found in commerce. Sensor array 408 provides pixel intensity values 410 of the captured IR image 409 of hand 402 to computer workstation 411. The computer workstation may be placed in communication with various components of the spectral measurement system 400 to control, for example, a focus of optics 405 and a sensitivity of detector array 408.

Workstation 411 is shown having a display 412 and keyboard 413 which collectively comprise a graphical user interface. The graphical user interface enables an operator or user of the system of FIG. 4 to enter or otherwise select one or more menu options and for modifying device settings. Alternatively, a touch screen display is utilized which enables the user to select menu options by physically touching the surface of the display 412. By using the graphical user interface, the user can define initial biological parameters, initiate various computational operations, and view results.

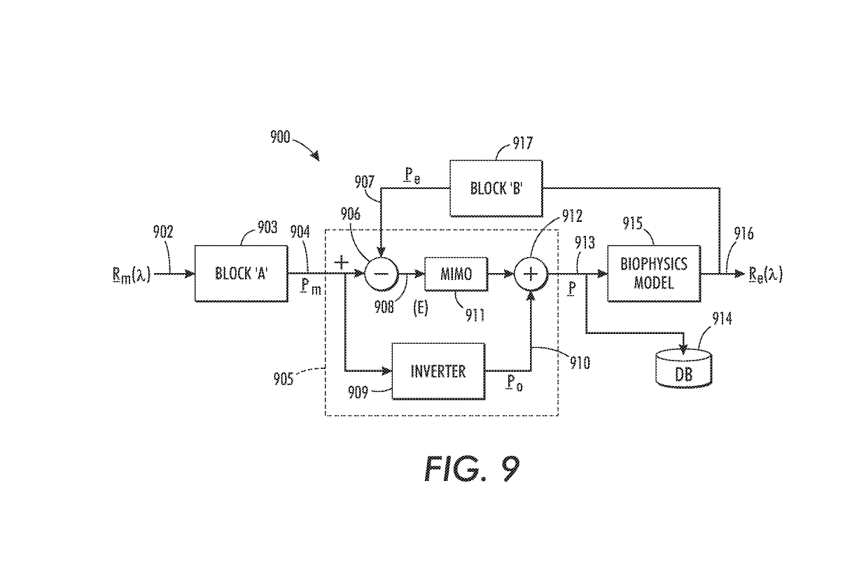

The workstation further comprises a computer case 414 housing a motherboard, CPU, memory, interface, storage device, and a communications link such as a network card. In this embodiment, workstation 411 is configured to receive signals of the captures IR images and perform various aspects of the teaching hereof as are further described with respect to the system of FIG. 9 such that a final virtual estimated virtual biological parameter vector can be generated and communicated to storage device 415 or to computer readable media 416.

It should be appreciated that workstation 411 necessarily includes a processor capable of executing machine readable program instructions to perform the functions described herein. Such functions include performing comparisons, computations, and the like. It should also be appreciated that workstation 411 includes machine executable instructions for displaying results onto display 412 and for communicating results over network 417 via wired or wireless communication pathways. Various components of the system of FIG. 4, individually or collectively, may comprise a special purpose computer system such as an ASIC or dedicated circuit. Computer 411 receives the measured spectrum 410 and processes those in accordance with the teachings hereof.

### Introduction to Diffuse Reflectance Spectroscopy

Diffuse reflectance spectroscopy consists of determining the radiative properties of an absorbing and scattering sample from diffuse reflectance measurements. In biological applications, the irradiated medium can be modeled as a strongly scattering multi-layer medium whose radiative properties are constant within each layer but differ from layer to layer. Skin consists of an outer layer called the epidermis and of an underlying layer called the dermis. As such, human skin can be modeled as a two-layer system. The epidermis is characterized by strong absorption in the ultraviolet and visible sections of the spectrum due to melanin content. The blood and connective tissues are responsible for absorption and scattering in the dermis. The absorption characteristics of blood depend on the concentrations of oxyhemoglobin and deoxyhemoglobin. The two-layer model enables human skin to be reasonably approximated as a finite epidermis overlaying a semi-infinite dermis. The hypodermis is assumed to diffuse all visible light because there are no chromospheres in subcutaneous fat. The two layer skin model can be used to relate skin properties to skin optical coefficients which, in turn, using for example a semi-empirical Kubelka-Munk model, can yield reasonably accurate estimates of the diffuse reflectance. Radiative properties, such as absorption and scattering, can be related to transmittance and reflectance spectra.

The fundamental equation governing photon transport is referred to as the Radiative Transfer Equation (RTE). One embodiment of the RTE is written as:

$$∇ {I}_{λ} ( \vec{r} , \hat{s} ) \cdot \hat{s} = {ɛ}_{λ} {I}_{λ} ( \vec{r} , \hat{s} ) - {σ}_{α , λ} {I}_{λ} ( \vec{r} , \hat{s} ) + {σ}_{s , λ} {I}_{λ} ( \vec{r} , \hat{s} ) + {σ}_{s , λ} {\int }_{4 π}^{ } {I}_{λ} ( \vec{r} , \hat{s} ) P ( {\hat{s}}_{i} , \hat{s} ) d {Ω}_{i} \qquad ( 4 )$$

where I_{λ} is the spectral intensity at location \vec{r} in direction ŝ. σ_{α,λ} and σ_{S,λ} are the absorption and scattering spectra. ε_{λ} is the emission spectra. The integral represents the light that is scattered in direction ŝ. P(ŝᵢ, ŝ) is the probability that a photon in direction ŝᵢ will be scattered in direction ŝ, and is referred to as the phase function. The RTE can be solved if appropriate boundary conditions at interfaces between media (e.g. air and skin) are accurately defined. These boundary conditions take the form of the well-known Snell's Law and Fresnel's Equations. The RTE can be solved using numerical methods. One such numerical method is the Monte Carlo method wherein absorption and scattering are treated as stochastic events which are modeled by sampling probability distributions for step size and angular defection. Monte Carlo is quite accurate once the structure (i.e., interfaces) and properties (i.e., absorption and scattering spectra) have been defined.

For planar geometries, RTE can be simplified sufficiently to yield analytical solutions. It can be shown that a single parameter, called the Effective Transport Albedo, can be used to describe photon transport. In one embodiment, this is given by:

$${w}_{tr} = \frac{{σ}_{s} ( 1 - g )}{{σ}_{α} + {σ}_{s} ( 1 - g )} \qquad ( 5 )$$

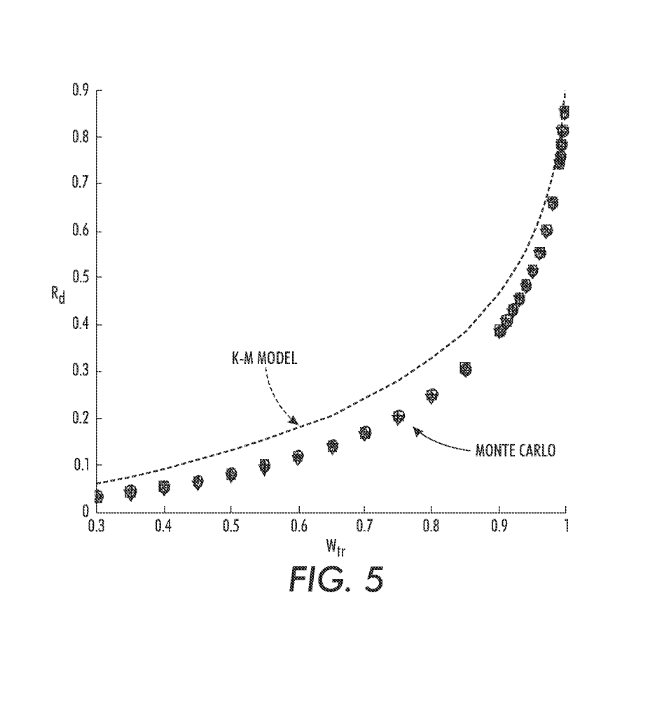

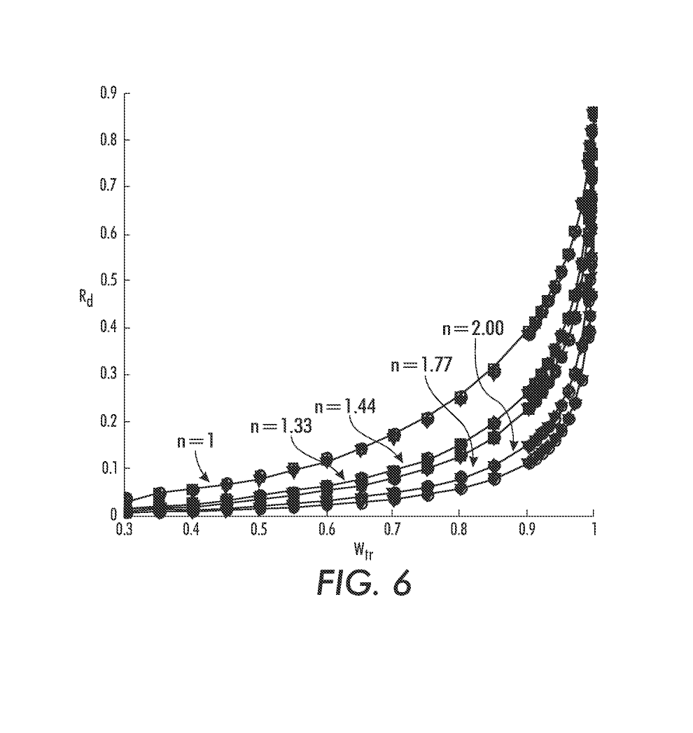

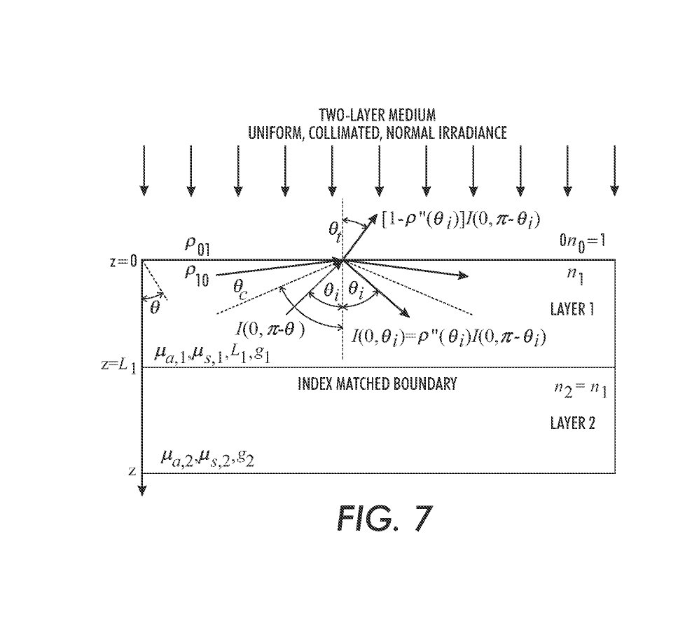

It can also be shown that the 1D RTE is essentially equivalent to the Kubelka-Munk (K-M) two flux model which is widely used to model color in printed images due to its computational efficiency. However, it should be appreciated that the K-M model is not all that accurate when compared to Monte Carlo. FIG. 5 shows a comparison of the K-M model to Monte Carlo for diffuse reflectances from a semi-infinite medium with a refractive index of n=1. FIG. 6 shows a comparison of the semi-empirical K-M model with Monte Carlo for a range of n where n is the refractive index of the layer. FIG. 7 shows a semi-empirical K-M model derived for a two layer geometry comprised of a thin (finite) top layer and a semi-infinite bottom layer.

In one embodiment, the two layer semi-empirical K-M model for diffuse reflectance from the surface on a finite layer is defined by:

R*(R_(wₜᵣ₁)−R_(wₜᵣ₂))+R_{W}_{ₜᵣ₂} (6)

where R_is the single layer semi-empirical K-M model given by:

$$\begin{matrix}R = ( 1 - {ρ}_{01} ) ( 1 - {\hat{ρ}}_{10} ) \frac{{\hat{R}}_{d}}{1 - {\hat{ρ}}_{10} {\hat{R}}_{d}} \\ where \text{:} & ( 7 ) \\ {\hat{ρ}}_{10} ( {η}_{1} , {w}_{tr} ) = {ρ}_{10} + {\sum }_{i = 0}^{N} {{A}_{i} ( {w}_{tr} )}^{i} & ( 8 ) \\ {\hat{R}}_{d } ( {η}_{1} , {w}_{tr} ) = {R}_{d} + {\sum }_{i = 0}^{N} {{B}_{i} ( {w}_{tr} )}^{i} & ( 9 )\end{matrix}$$

where ρ₀₁ is the specular reflectance, ρ₁₀ is the surface reflectance from media to air, and R_{d} is the K-M diffuse reflectance given by: R_{d}=a−\sqrt{a²−1}, where a is the K-M parameters expressed as a function of wₜᵣ. Quantities denoted with the symbol ‘̂’ refer to empirically modified quantities with the empirical coefficients Aᵢ and Bᵢ obtained from regression fits to the Monte Carlo results. The parameter R* is a matching parameter given by:

$${R}^{*} = \frac{tanh ( Y )}{1 / α + ( 1 - \frac{1}{α} ) tanh ( Y )} \qquad ( 10 )$$

where Y is the K-M optical thickness of the top layer and a is an empirical factor which is a function of wₜᵣ₂ such that:

$$1 / \hat{α} = {\sum }_{i = 0}^{M} {{C}_{i} ( {w}_{tr 2} )}^{i} \qquad ( 11 )$$

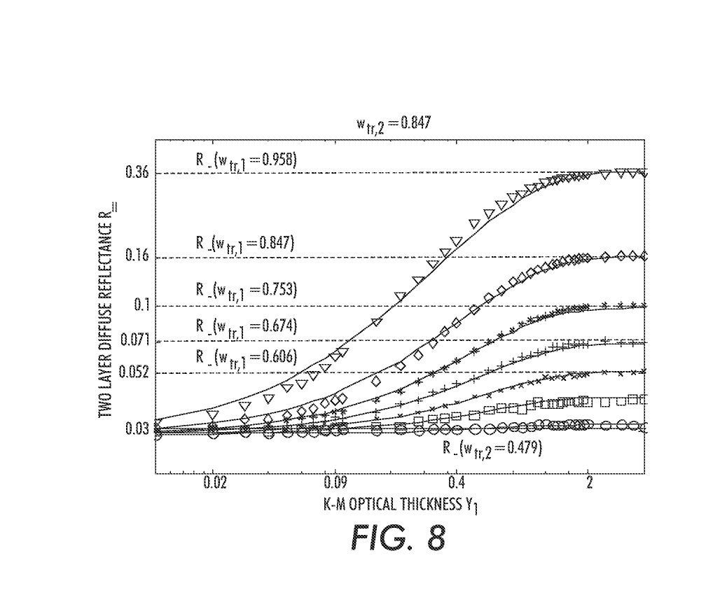

FIG. 8 shows a comparison of the semi-empirical K-M to Monte Carlo for the two layer structure of FIG. 7.

### Skin Parameter (Property) Vector:

The skin models maps skin properties to optical properties of the skin layers which can then be used to calculate reflectance spectra using a semi-empirical K-M model.

In general, the absorption σₐ and scattering spectra σₛ in the skin model take the form:

σ_{a,i}=f(p),σ_{s,i}=g(p) (12)

where p is a vector of skin properties, and f and g are mapping functions that map these properties to optical properties of skin layer i. Details of skin models in the visible to NIR can be found in the above-incorporated references entitled: “Simple And Accurate Expressions For Diffuse Reflectance Of A Semi-Infinite And Two-Layer Absorbing And Scattering Media” and “Retrieving Skin Properties From In Vivo Spectral Reflectance Measurements”, by Yudovsky and Pilon. In this range, the two-layer semi-empirical K-M model is of particular interest because skin can be reasonably approximated as a finite epidermis overlaying a semi-infinite dermis layer. In various embodiments hereof, skin parameters are given by a vector:

p=[Lₑₚᵢ,fₘₑₗ,f_{blood},SO₂,C_{S}]^{T} (13)

where Lₑₚᵢ is the epidermis layer thickness, fₘₑₗ is the melanin concentration in the epidermis, f_{blood} is the volume fraction of blood in the dermis layer, SO₂ is oxygen saturation in the blood, and C_{S} is a light scattering parameter. T is a symbol used to represent a transpose operation. The refractive index of both the dermis and epidermis has been found to be ≈1.44 and the scattering anisotropy parameter in both layers (g) can be well approximated by ≈0.77. The accuracy of the semi-empirical K-M model is relatively insensitive to g.

The absorption spectra in the epidermis can be expressed in terms of:

σ_{a,epi}(λ)=σ_{a,mel}fₘₑₗ+σ_{a,bkg}(1−fₘₑₗ) (14)

where σ_{a,mel} is the melanin extinction spectra and σ_{a,bkg} is the background absorption spectra where σ_{a,bkg}=7.84×10⁸λ^{-3.255}.

Similarly, the absorption spectra in the dermis can be expressed in terms of:

σ_{a,derm}(λ)=σ_{a,blood}f_{blood}+σ_{a,bkg}(1−f_{blood}) (15)

where σ_{a,blood} is the absorption spectra of blood which can be further expressed as: σ_{a,blood}=σ_{a,oxy}+σ_{a,deoxy}, where σ_{a,oxy} and σ_{a,deoxy} is the absorption spectra of the oxygenated and de-oxygenated blood, respectively, as defined by:

$$\begin{matrix}{σ}_{a , oxy} = \frac{{ɛ}_{oxy} ( λ ) {C}_{heme} {SO}_{2}}{64 , 500} & ( 16 ) \\ {σ}_{a , deoxy} = \frac{{ɛ}_{deoxy} ( λ ) {C}_{heme} ( 1 - {SO}_{2} )}{64 , 500} & ( 17 )\end{matrix}$$

where ε_{oxy} and ε_{deoxy} are the molar extinction coefficients of oxygenated and deoxygenated hemoglobin, respectively, and Cₕₑₘₑ is the concentration of hemoglobin in blood which is typically 150 g/liter. The scattering spectra in both the dermis and epidermis is: σ_{S}(λ)=C_{S}×10⁵λ^{-1.30} with C_{S}=5×10⁵. This completes the description of the two-layer skin model.

### Block Diagram of Inversion

Reference is now being made to FIG. 9 which shows one embodiment of a block diagram of a system 900 for the inversion of the skin model. Given an initial parameter vector p, of Eq. (13), estimated reflectance spectra can be generated. An iterative control-based refinement approach is used to further improve the accuracy of the estimated virtual biological parameter vector. Iterations are performed on the skin model by comparing the measured virtual parameter vector Pₘ and the estimated virtual parameter vector Pₑ followed by processing the error to generate a new estimated virtual biological parameter vector which is used on a next iteration. When the iterative approach converges, the norm of the error vector calculated between Pₘ and Pₑ will be close to zero, or may start to go higher.

In FIG. 9, measured spectrum Rₘ(λ) (at 902) are provided to Block ‘A’ (at 903) wherein the measured spectrum are transformed to a lower dimensional virtual parameters using, for example, a least squares method to obtain a measured virtual biological parameter vector pₘ (at 904). The measured virtual biological parameter vector 904 is provided to the inversion algorithm, shown generally at 905, wherein the measured virtual biological parameter vector 904 is provided to comparator 906 which compares that against an estimated virtual biological parameter vector pₑ (at 907) to determine an error E (at 908) therebetween. The measured virtual biological parameter vector 904 is also provided to an inverter 909 which uses a Genetic Algorithm (GA) to derive an initial biological parameter vector P₀ (at 910). Constrained-LMA or SPSA can also be used within module 909. The determined error 908 is provided to controller 911 which is added (at 912) to the obtained vector 910 to generate a next estimated virtual biological parameter vector P (at 913). The next estimated virtual biological parameter vector 913 is stored to storage device 914. It should be appreciated that, on a first iteration, the initial biological parameter vector 910 is provided, as input, to biophysics model 915 to obtain estimated spectrum Rₑ(λ) (at 916). The controller is preferably designed such that convergence is achieved at a near-zero value.

In the embodiment of FIG. 9, the feedback controller 911 comprises a MIMO (multi-input multi-output) integral controller with a gain matrix K which has been designed using a pole-placement strategy or LQR (Linear Quadratic Regulator) by computing the Jacobian at the nominal values of the biological parameter vector. Such methods are described in the above-incorporated reference entitled: “Control of Color Imaging Systems Analysis and Design”, CRC Press, (May 2009), ISBN 978-0849337468. It should be noted that, during iterations, the estimated spectrum Rₑ(λ) will exactly match the measured spectrum Rₘ(λ) when the norm of the error vector is zero; provided, of course, that the number basis used fully approximates the biophysics model. In reality, an exact match between the two spectrum will not be achieved due to limitations which include, for instance, noise in the measured spectrum. Iterations are preferably carried out until the norm of the error vector is at or below an acceptable user-defined threshold level. The iteration history is preferably stored. Methods for such iterative approach and picking the best final parameter vector is described in Section 7.5.2.1 of the above-incorporated text entitled: “Control of Color Imaging Systems: Analysis and Design”, CRC Press (2009), ISBN-13: 9780849337468.

The generated estimated reflectance spectrum 916 is provided to Block ‘B’ (at 917) wherein the parameters are transformed to a lower dimensional virtual parameter space represented by pₑ (at 907). It should be appreciated that the transformation to a lower dimensional parameter space that occurs in Block ‘A’ is the same as the transformation that occurs in Block ‘B’. As such, in other embodiments, Block ‘A’ and Block ‘B’ are combined into a single Block.

The system of FIG. 9 is an iterative process which repeats until convergence of a minimum error. Upon convergence, the last biological parameter vector 913 that was stored in storage device 915 is determined to be the final estimated virtual biological parameter vector P_{F}.

### Formal Derivation

Let ψ(λ) denote a matrix containing basis functions. Construct a natural basis set by performing design of experiments (DOE) on either the biophysics model or on a Monte Carlo simulator. It should be appreciated that other mathematical basis functions (e.g., wavelet, DCT, etc.) can also be used. The natural basis set is preferable since it can lead to significantly lower dimensional virtual parameters.

For a general estimated spectrum Rₑ(λ), let the estimated virtual biological parameter vector pₑ=[p₁ p₂ p₃ . . . p_{N}]^{T} where Pᵢ is the i^{tb} parameter, T denotes a transpose operation, and N is the number of parameters. The equation for the estimated spectrum can be derived in terms of the natural basis set as follows:

$$\begin{matrix}{R}_{e} ( λ ) = {\sum }_{i = 1}^{N} {P}_{i} {\underline{Ψ}}_{i} ( λ ) \\ = [ {\underline{ψ}}_{1} ( λ ) , {\underline{ψ}}_{2} ( λ ) , … , {\underline{ψ}}_{N} ( λ ) ] [ \begin{matrix}{P}_{1} \\ {P}_{2} \\ ⋮ \\ {P}_{N}\end{matrix} ] \\ = ψ ( λ ) {\underline{p}}_{e}\end{matrix} \qquad ( 18 )$$

Multiplying both sides of Eq. (14) by ψ^{T}(λ) and rearranging terms, we get:

Pₑ=[ψ^{T}(λ)ψ(λ)]⁻¹ψ^{T}(λ)Rₑ(λ) (19)

Similarly, for the output of Block A, we get.

Pₘ=[ψ^{T}(λ)ψ(λ)]⁻¹ψ^{T}(λ)Rₘ(λ) (20)

### Flow Diagram of One Embodiment

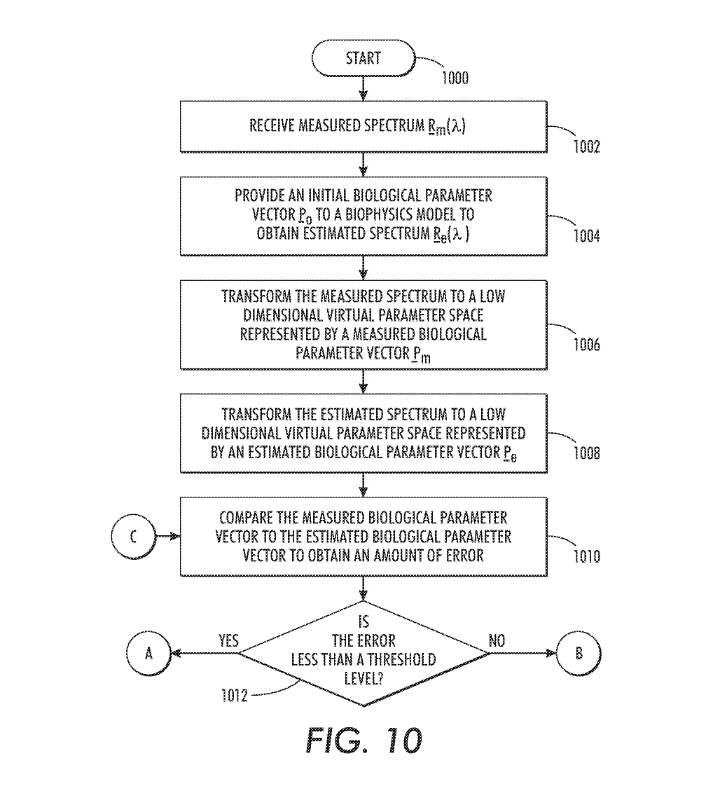

Reference is now being made to the flow diagram of FIG. 10 which illustrates one embodiment of the present method for estimating a biological parameter vector for a biophysics model from reflectance measurements obtained from a reflectance-based spectral measurement system. Flow processing begins at step 1000 and immediately proceeds to step 1002.

At step 1002, receive measured spectrum Rₘ(λ). The received measured spectrum comprise in-vivo spectral reflectance measurements obtained by a spectral reflectance sensing device at wavelength λ from a surface of a biological entity. The surface of the biological entity is represented by a biophysics model for which a biological parameter vector P is to be estimated.

At step 1004, provide an initial biological parameter vector P₀ to the biophysics model to obtain estimated spectrum Rₑ(λ)

At step 1006, transform the measured spectrum Rₘ(λ) to a low dimensional virtual parameter space represented by a measured virtual biological parameter vector Pₘ.

At step 1008, transform the estimated spectrum Rₑ(λ) to a low dimensional virtual parameter space represented by an estimated virtual biological parameter vector Pₑ.

At step 1010, compare the measured virtual biological parameter vector to the estimated virtual biological parameter vector to obtain an amount of an error vector E.

At step 1012, a determination is made whether the error vector (of step 1010) is less than a pre-defined threshold.

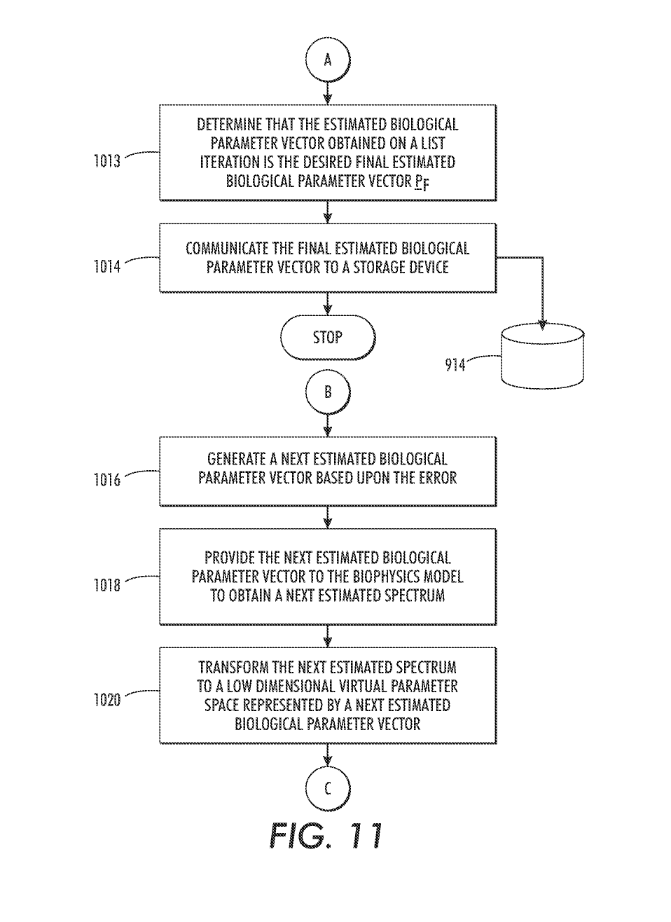

Reference is now being made to the flow diagram of FIG. 11 which is a continuation of the flow diagram of FIG. 10.

If, as a result of the determination of step 1012, the error is less than the pre-defined threshold level then processing continues with respect to node A wherein, at step 1013, determine that the last estimated virtual biological parameter vector is the desired final estimated virtual biological parameter vector P_{F}. At step 1014, communicate the final estimated virtual biological parameter vector P_{F} to a storage device such as, for example, storage device 914 of FIG. 9. Thereafter, in this embodiment, further processing stops. On the other hand, if, as a result of the determination of step 1012, the error is determined to be greater than or equal to the pre-defined threshold level then processing continues with respect to node B wherein, at step 1016, generate a next estimated virtual biological parameter vector based upon the determined amount of error.

At step 1018, provide the next estimated virtual biological parameter vector to the biophysics model to obtain a next estimated spectrum.

At step 1020, transform the next estimated spectrum to a low dimensional virtual parameter space represented by a next estimated virtual biological parameter vector Pₑ. Processing thereafter continues with respect to node C wherein, at step 1010, the measured virtual biological parameter vector is compared to this next estimated virtual biological parameter vector to obtain an amount of an error vector E. Processing repeats in such a manner until the error is determined to be below a desired threshold level.

It should be appreciated that the flow diagrams hereof are illustrative. One or more operative may be added, modified or enhanced. Such variations are intended to fall within the scope of the appended claims. All or portions of the flow diagrams may be implemented partially or fully in hardware in conjunction with machine executable program instructions.

### Performance Results

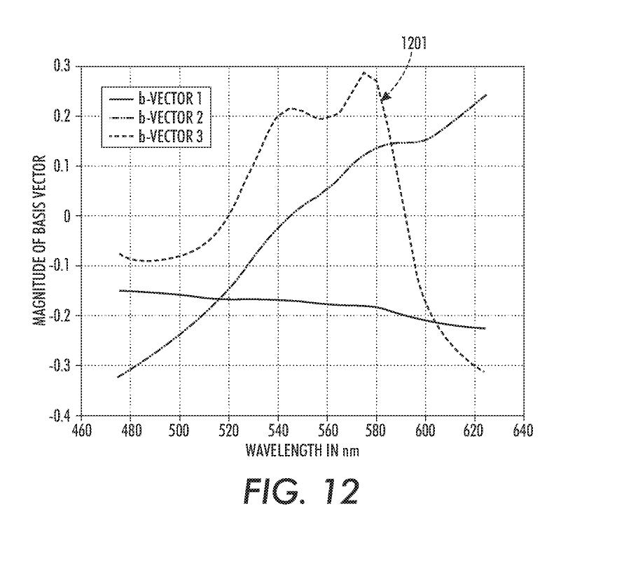

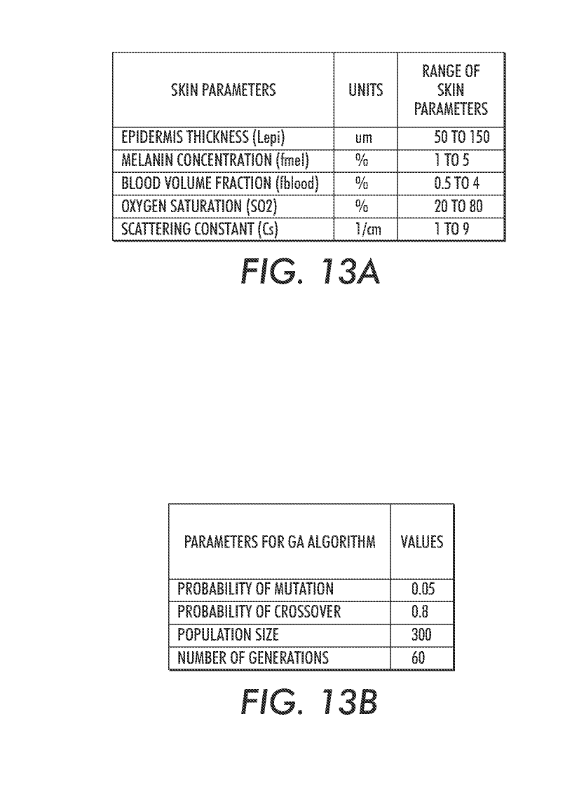

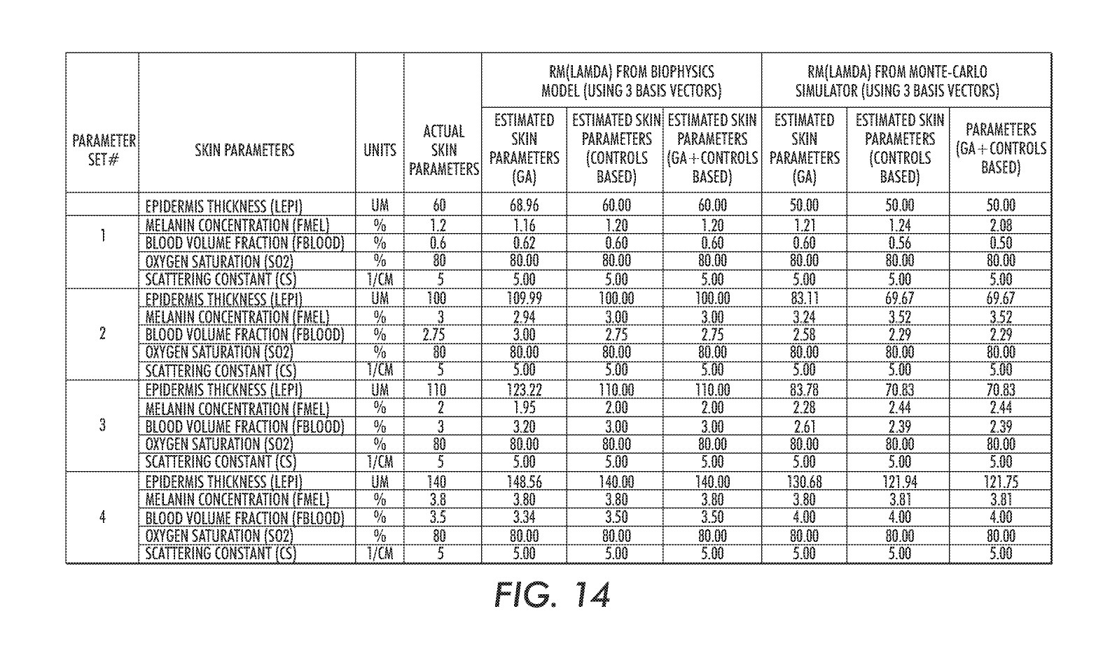

Test spectra (at 1201 in FIG. 12) were created. Genetic Algorithms (GA) were used to derive a set of initial biological parameters. Values used are provided in the tables of FIGS. 13A-B. Basis vectors were constructed using the biophysics model for the range of skin parameters shown in FIG. 14. Only three basis vectors were used in our simulation. A 3D input-output model was obtained between the initial biological parameter vector P and the estimated virtual biological parameter vector Pₑ. The Jacobian was calculated at Pₒ. The gain matrix was obtained using the Jacobian and the MIMO pole placement algorithm. Spectra were obtained from the biophysics model and a Monte-Carlo simulator for four different parameter sets. Since only three basis vectors were considered, only three parameters were estimated for Rₘ(λ). Results are also shown in FIG. 14. For simulation without GA, nominal values at the mid-point were used. As shown, the estimated virtual biological parameter values generated were remarkable.

### Example Functional Block Diagram

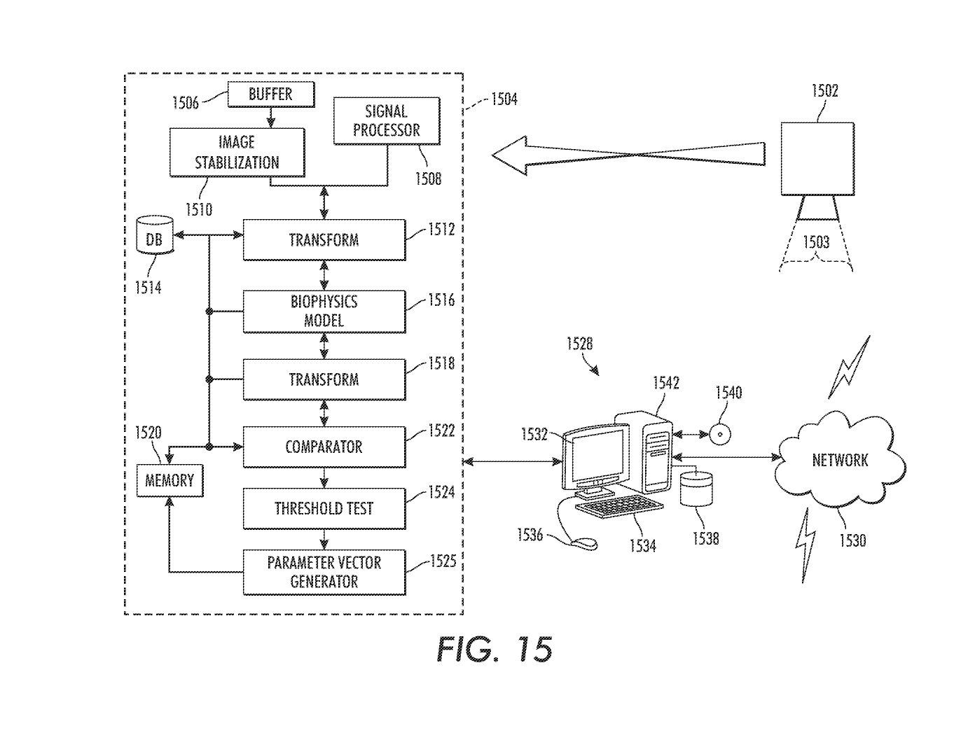

Reference is now being made to FIG. 15 which illustrates a block diagram of one example processing system for implementing various aspects of the present method described with respect to the flow diagrams of FIGS. 10 and 11, and the iterative system of FIG. 9.

In FIG. 15, spectral reflectance sensing device 1502 captures one or more IR images of an area of exposed skin of a subject of interest placed in the device's field of view 1503. Various embodiments of the spectral measurement device 1502 may comprise some or all of the features and functionality shown and discussed with respect to the system of FIG. 4. The captured image data are communicated to image processing system 1504. In the embodiment of FIG. 15, the image processing system comprises a Buffer 1506 for queuing received image data. Buffer 1506 may further store mathematical formulas and representations as necessary to process the received image data in accordance with various embodiments hereof. Signal Processor 1508 processes the pixel intensity values to remove noise. Image Stabilizer 1510 is shown for completeness for those embodiments where noise from either the motion of the spectral measurement system or movement of the subject is to be compensated. Images can be stabilized using, for example, image segmentation and point feature tracking. Such techniques are well known in the image processing arts.

The measured spectrum are provided to Transform Module 1512 which transforms the measured spectrum to a low dimensional virtual parameter space represented by a measured virtual biological parameter vector Pₘ and stores the values to storage device 1514. Biophysics Model 1516 receives initial biological parameter vector P₀ and generates an estimated spectrum Rₑ(λ). Various aspects of the biophysics model may also be retrieved from storage device 1514. Transform Module 1518 transforms the estimated spectrum to a low dimensional virtual parameter space represented by an estimated virtual biological parameter vector Pₑ. Comparator 1522 performs a comparison between the measured virtual biological parameter vector Pₘ and the estimated virtual biological parameter vector Pₑ to determine an error E therebetween. The determined error is stored in Memory 1520. Threshold Test Processor 1524 determines whether the error is less than a pre-defined threshold. If so then the last estimated virtual biological parameter vector is determined to be the final estimated virtual biological parameter vector P_{F}. The final estimated virtual biological parameter vector is communicated to workstation 1528 where these virtual parameters and various results are displayed on the display device thereof. Such results may take the form of one or more aspects of the Table of FIG. 14. If Threshold Test 1524 determines whether the error is not less than a pre-defined threshold, then Parameter Vector Generator 1525 generates a next biological parameter vector based upon the determined amount of error. The next biological parameter vector is communicated or otherwise provided to the Biophysics Model 1516 to obtain a next estimated spectrum. In this embodiment, the various modules communicate via Memory 1520 wherein values are stored and retrieved. The next estimated spectrum is transformed (at 1518) to a low dimensional virtual parameter space represented by a next estimated virtual biological parameter vector. The process of FIG. 15 iteratively repeats until Threshold Test Module 1524 determines that the error is below an acceptable level, at which point, the final estimated virtual biological parameter vector P_{F} is communicated to workstation 1528 and further provide to storage device 1538.

It should be appreciated that some or all of the functionality performed by any of the modules or processing units of the system of FIG. 15 can be performed, in whole or in part, by the computer workstation. Workstation 1528 is placed in communication with network 1530 via a communications interface (not shown). The workstation of FIG. 15 is shown comprising a display 1532 for displaying information and for effectuating a user input or selection such as, for example, the user providing an initial biological parameter vector. Display 1532 may be placed in communication with any of the modules and processors of the system 1504 and/or the measurement device 1502 such that images and spectral measurements obtained thereby can be viewed on the display device. A user or technician of the system of FIG. 15 may use the graphical user interface of workstation 1528, e.g., keyboard 1534 and mouse 1536, to identify regions of interest, set parameters and enter values, select pixels, frames, images, and/or regions of images for processing. Data entered and selection made by the user may be stored to storage medium 1538 or to computer readable media 1540.

It should be appreciated that the workstations of FIGS. 4 and 15 have an operating system and other specialized software configured to display a variety of numeric values, text, scroll bars, pull-down menus with user selectable options, and the like, for entering, selecting, or modifying information displayed thereon. Information stored to a computer readable media can be retrieved by a media reader such as, for example, a CD-ROM or DVD drive. Any of the modules and processing units of FIG. 15 can be placed in communication with database 1538 and may store/retrieve therefrom data, variables, records, parameters, functions, machine readable/executable program instructions required to perform their intended functions. Moreover each of the modules of the processing system 1504 may be placed in communication with one or more devices over network 1530.

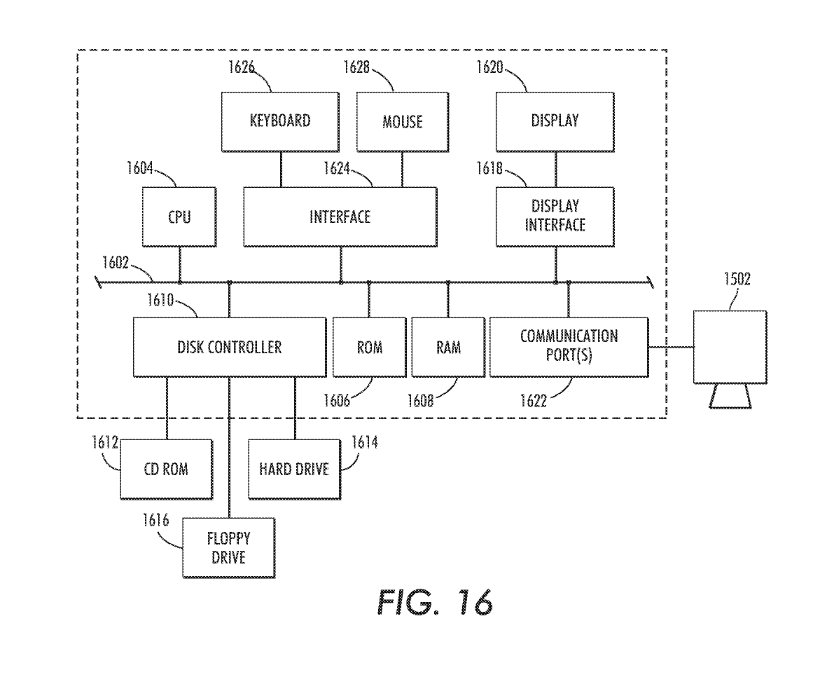

It should also be appreciated that various modules may designate one or more components which may, in turn, comprise software and/or hardware designed to perform the intended function. A plurality of modules may collectively perform a single function. Each module may have a specialized processor capable of executing machine readable program instructions. A module may comprise a single piece of hardware such as an ASIC, electronic circuit, or special purpose processor such as that which is shown and discussed with respect to the embodiment of FIG. 16. A plurality of modules may be executed by either a single special purpose computer system or a plurality of special purpose computer systems operating in parallel. Connections between modules include both physical and logical connections. Modules may further include one or more software/hardware modules which may further comprise an operating system, drivers, device controllers, and other apparatuses some or all of which may be connected via a network.

### Example Special Purpose Computer

Reference is now being made to FIG. 16 which illustrates a block diagram of one example special purpose computer for implementing one or more aspects of the present method. Such a special purpose processor is capable of executing machine executable program instructions and may comprise any of a micro-processor, micro-controller, ASIC, electronic circuit, or any combination thereof.

In FIG. 16, communications bus 1602 is in communication with a central processing unit (CPU) 1604 capable of executing machine readable program instructions for performing any of the calculations, comparisons, logical operations, and other program instructions for performing any of the steps described above with respect to the flow diagrams and illustrated embodiments hereof. Processor 1604 is in communication with memory (ROM) 1606 and memory (RAM) 1608 which, collectively, constitute example storage devices. Such memory may be used to store machine readable program instructions and other program data and results to sufficient to carry out any of the functionality described herein. Disk controller 1610 interfaces with one or more storage devices 1614 which may comprise external memory, zip drives, flash memory, USB drives, or other devices such as CD-ROM drive 1612 and floppy drive 1616. Storage device stores machine executable program instructions for executing the methods hereof. Such storage devices may be used to implement a database wherein various records are stored. Display interface 1618 effectuates the display of information on display 1620 in various formats such as, for instance, audio, graphic, text, and the like. Interface 1624 effectuates a communication via keyboard 1626 and mouse 1628, collectively a graphical user interface. Such a graphical user interface is useful for a user to enter information about any of the displayed information in accordance with various embodiments hereof. Communication with external devices may occur using example communication port(s) 1622. One such external device placed in communication with the special purpose computer system of FIG. 16 is the spectral sensing measurement device 1502 of FIG. 15. Such ports may be placed in communication with any of the modules and components of the example networked configuration of FIGS. 4 and 15, as shown and described herein, using the Internet or an intranet, either by direct (wired) link or wireless link. Example communication ports include modems, network cards such as an Ethernet card, routers, a PCMCIA slot and card, USB ports, and the like, capable of transferring data from one device to another. Software and data is transferred via the communication ports in the form of signals which may be any of digital, analog, electromagnetic, optical, infrared, or other signals capable of being transmitted and/or received by the communications interface. Such signals may be implemented using, for example, a wire, cable, fiber optic, phone line, cellular link, RF, or other signal transmission means presently known in the arts or which have been subsequently developed.

It will be appreciated that the above-disclosed and other features and functions, or alternatives thereof, may be desirably combined into many other different systems or applications. Various presently unforeseen or unanticipated alternatives, modifications, variations, or improvements therein may become apparent and/or subsequently made by those skilled in the art which are also intended to be encompassed by the following claims. Accordingly, the embodiments set forth above are considered to be illustrative and not limiting. Various changes to the above-described embodiments may be made without departing from the spirit and scope of the invention.

The teachings hereof can be implemented in hardware or software using any known or later developed systems, structures, devices, and/or software by those skilled in the applicable art without undue experimentation from the functional description provided herein with a general knowledge of the relevant arts. Moreover, the methods hereof can be implemented as a routine embedded on a personal computer or as a resource residing on a server or workstation, such as a routine embedded in a plug-in, a driver, or the like. Furthermore, the teachings hereof may be partially or fully implemented in software using object or object-oriented software development environments that provide portable source code that can be used on a variety of computer, workstation, server, network, or other hardware platforms. One or more of the capabilities hereof can be emulated in a virtual environment as provided by an operating system, specialized programs or leverage off-the-shelf computer graphics software such as that in Windows, Java, or from a server or hardware accelerator or other image processing devices.

One or more aspects of the methods described herein are intended to be incorporated in an article of manufacture, including one or more computer program products, having computer usable or machine readable media. The article of manufacture may be included on a storage device readable by a machine architecture embodying executable program instructions capable of performing the methodologies described herein. The article of manufacture may be included as part of a standalone system, an operating system, or a software package which may be shipped, sold, leased, or otherwise provided either alone or as part of an add-on, update, upgrade, or product suite. It will be appreciated that various features and functions and alternatives hereof may be combined into other systems or applications which are heretofore unknown.

Various presently unforeseen or unanticipated alternatives, modifications, variations, or improvements therein may become apparent and/or subsequently made by those skilled in the art which are also intended to be encompassed by the following claims. Accordingly, the embodiments set forth above are considered to be illustrative and not limiting. Changes to the above-described embodiments may be made without departing from the spirit and scope of the invention. The teachings of any printed publications including patents and patent applications, are each separately hereby incorporated by reference in their entirety.

What is claimed is:

1. A method for estimating a biological parameter vector for a biophysics model from reflectance measurements obtained from a reflectance-based spectral measurement system, the method comprising:
providing, as input, an initial biological parameter vector P₀ to said biophysics model, said biophysics model generating, as output, an estimated spectrum Rₑ(λ)
transforming said estimated spectrum to a low dimensional virtual parameter space represented by an estimated virtual biological parameter vector Pₑ; and
communicating said estimated virtual biological parameter vector to a storage device.

2. The method of claim 1, further comprising:
receiving measured spectrum Rₘ(λ) comprising in-vivo spectral reflectance measurements obtained by a spectral reflectance sensing device at wavelength λ from a surface of a biological entity, said surface being represented, in part, by a biophysics model for which a biological parameter vector P is to be estimated; and
transforming said measured spectrum to a low dimensional virtual parameter space represented by a measured virtual biological parameter vector Pₘ.

3. The method of claim 2, further comprising:
(A) comparing said measured virtual biological parameter vector Pₘ to said estimated virtual biological parameter vector Pₑ to determine an error E therebetween;
(B) in response to said error being less than a pre-defined threshold, determining that a last estimated virtual biological parameter vector to be a final estimated virtual biological parameter vector P_{F}, otherwise comprising:
(i) generating a next biological parameter vector based upon said determined amount of error;
(ii) providing said next biological parameter vector to said biophysics model to obtain a next estimated spectrum;
(ii) transforming said next estimated spectrum to a low dimensional virtual parameter space represented by a next estimated virtual biological parameter vector, said next estimated virtual biological parameter vector being used on a next iteration; and
repeating (A)-(B); and
communicating said final estimated virtual biological parameter vector to said storage device.

4. The method of claim 1, wherein said spectral reflectance sensing device comprises any of: a spectrometer, a spectrophotometer, a multi-spectral camera, and a hyperspectral camera.

5. The method of claim 1, wherein said estimated spectrum Rₑ(λ) comprises:
$${\underline{R}}_{e} ( λ ) = {\sum }_{i = 1}^{N} {P}_{i} {\underline{Ψ}}_{i} ( λ )$$
where Pᵢ is the i^{tb} parameter in said biological parameter vector, ψ₁(λ), ψ₂(λ), ψ₃(λ), . . . , ψ_{N}(λ) represent column-wise basis vectors 1, 2, 3, . . . N, respectively, with each element along a row representing a basis value for wavelength λ and N is the number of parameters.

6. The method of claim 1, wherein said estimated virtual biological parameter vector Pₑ comprises:
Pₑ=[ψ^{T}(λ)ψ(λ)]⁻¹ψ^{T}(λ)Rₑ(λ)
where ψ(λ) is a matrix with column-wise basis vectors with each element representing a basis value for wavelength λ, and T is a transpose operation.

7. The method of claim 1, wherein said measured virtual biological parameter vector Pₘ comprises:
Pₘ=[ψ^{T}(λ)ψ(λ)]⁻¹ψ^{T}(λ)Rₘ(λ)
where ψ(λ) is a matrix with column-wise basis vectors with each element representing a basis value for wavelength λ, and T is a transpose operation.

8. The method of claim 1, wherein said initial biological parameter vector P₀ is generated using any of: a Genetic Algorithm, a Constrained Levenberg-Marquard algorithm, and a Simultaneous Perturbation Stochastic Approximation.

9. The method of claim 1, wherein said next estimated virtual biological parameter vector is determined using a feedback controller comprising a MIMO (multi-input multi-output) integral controller with a gain matrix K designed using any of: a pole-placement strategy, and Linear Quadratic Regulator (LQR) by having computed a Jacobian matrix at nominal values of said biological parameter vector.

10. The method of claim 1, wherein said biophysics model comprises a model of multi-layered skin tissue, said biological parameter vector comprising any combination of: epidermal thickness, melanin concentration, dermal blood volume fraction, oxygen saturation, and a light scattering parameter.

11. A system for estimating a biological parameter vector for a biophysics model from reflectance measurements obtained from a reflectance-based spectral measurement device, the system comprising:
a spectral reflectance sensing device for obtaining in-vivo spectral reflectance measurements at wavelength λ from a surface of a biological entity, said surface being represented, in part, by a biophysics model for which a biological parameter vector P is to be estimated;
a processor in communication with a storage device and said spectral reflectance sensing device, said process executing machine readable program instructions for performing:
receiving an initial biological parameter vector P₀ into said biophysics model to generate an estimated spectrum Rₑ(λ);
transforming said estimated spectrum to a low dimensional virtual parameter space represented by an estimated virtual biological parameter vector Pₑ; and
communicating said estimated virtual biological parameter vector to said storage device.

12. The system of claim 11, further comprising:
receiving measured spectrum Rₘ(λ) comprising in-vivo spectral reflectance measurements obtained by a spectral reflectance sensing device at wavelength λ from a surface of a biological entity, said surface being represented, in part, by a biophysics model for which a biological parameter vector P is to be estimated; and
transforming said measured spectrum to a low dimensional virtual parameter space represented by a measured virtual biological parameter vector Pₘ.

13. The system of claim 12, further comprising:
(A) comparing said measured virtual biological parameter vector Pₘ to said estimated virtual biological parameter vector Pₑ to determine an error E therebetween;
(B) in response to said error being less than a pre-defined threshold, determining that a last estimated virtual biological parameter vector to be a final estimated virtual biological parameter vector P_{F}, otherwise comprising:
(i) generating a next biological parameter vector based upon said determined amount of error;
(ii) providing said next biological parameter vector to said biophysics model to obtain a next estimated spectrum;
(ii) transforming said next estimated spectrum to a low dimensional virtual parameter space represented by a next estimated virtual biological parameter vector, said next estimated virtual biological parameter vector being used on a next iteration; and
repeating (A)-(B); and
communicating said final estimated virtual biological parameter vector to said storage device.

14. The system of claim 11, wherein said spectral reflectance sensing device comprises any of: a spectrometer, a spectrophotometer, a multi-spectral camera, and a hyperspectral camera.

15. The system of claim 11, wherein said estimated spectrum Rₑ(λ) comprises:
$${\underline{R}}_{e} ( λ ) = {\sum }_{i = 1}^{N} {P}_{i} {\underline{Ψ}}_{i} ( λ )$$
where Pᵢ is the i^{tb} parameter in said biological parameter vector, ψ₁(λ), ψ₂(λ), ψ₃(λ), . . . , ψ_{N}(λ) represent column-wise basis vectors 1, 2, 3, . . . N, respectively, with each element along a row representing a basis value for wavelength λ and N is the number of parameters.

16. The system of claim 11, wherein said estimated virtual biological parameter vector Pₑ comprises:
Pₑ=[ψ^{T}(λ)ψ(λ)]⁻¹ψ^{T}(λ)Rₑ(λ)
where ψ(λ) is a matrix with column-wise basis vectors with each element representing a basis value for wavelength λ, and T is a transpose operation.

17. The system of claim 11, wherein said measured virtual biological parameter vector Pₘ comprises:
Pₘ=[ψ^{T}(λ)ψ(λ)]⁻¹ψ^{T}(λ)Rₘ(λ)
where ψ(λ) is a matrix with column-wise basis vectors with each element representing a basis value for wavelength λ, and T is a transpose operation.

18. The system of claim 11, wherein said initial biological parameter vector P₀ is generated using any of: a Genetic Algorithm, a Constrained Levenberg-Marquard algorithm, and a Simultaneous Perturbation Stochastic Approximation.

19. The system of claim 11, wherein said next estimated virtual biological parameter vector is determined using a feedback controller comprising a MIMO (multi-input multi-output) integral controller with a gain matrix K designed using any of: a pole-placement strategy, and Linear Quadratic Regulator (LQR) by having computed a Jacobian matrix at nominal values of said biological parameter vector.

20. The system of claim 11, wherein said biophysics model comprises a model of multi-layered skin tissue, said biological parameter vector comprising any combination of: epidermal thickness, melanin concentration, dermal blood volume fraction, skin oxygen saturation, and a light scattering parameter.

21. A computer implemented method for estimating a biological parameter vector for a biophysics model from reflectance measurements obtained from a reflectance-based spectral measurement system, the method comprising:
receiving measured spectrum Rₘ(λ) comprising in-vivo spectral reflectance measurements obtained by a spectral reflectance sensing device at wavelength λ from a surface of a biological entity, said surface being represented, in part, by a biophysics model for which a biological parameter vector P is to be estimated;
providing, as input, an initial biological parameter vector P₀ to said biophysics model, said biophysics model generating, as output, an estimated spectrum Rₑ(λ);
transforming said measured spectrum Rₘ(λ) to a low dimensional virtual parameter space represented by a measured virtual biological parameter vector Pₘ;
transforming said estimated spectrum Rₑ(λ) to a low dimensional virtual parameter space represented by an estimated virtual biological parameter vector Pₑ;
(A) comparing said measured virtual biological parameter vector to said estimated virtual biological parameter vector to determine an error E therebetween;
(B) in response to said error being less than a pre-defined threshold, determining that a last estimated virtual biological parameter vector to be a final estimated virtual biological parameter vector P_{F}, otherwise comprising:
(i) generating a next biological parameter vector based upon said determined amount of error;
(ii) providing said next biological parameter vector to said biophysics model to obtain a next estimated spectrum;
(ii) transforming said next estimated spectrum to a low dimensional virtual parameter space represented by a next estimated virtual biological parameter vector, said next estimated virtual biological parameter vector being used on a next iteration; and
repeating (A)-(B); and
communicating said final estimated virtual biological parameter vector P_{F} to a storage device.

22. The computer implemented method of claim 21, wherein said estimated spectrum Rₑ(λ) comprises:
$${\underline{R}}_{e} ( λ ) = {\sum }_{i = 1}^{N} {P}_{i} {\underline{Ψ}}_{i} ( λ )$$
where Pᵢ is the i^{tb} parameter in said biological parameter vector, ψ₁(λ), ψ₂(λ), ψ₃(λ), . . . , ψ_{N}(λ) represent column-wise basis vectors 1, 2, 3, . . . N, respectively, with each element along a row representing a basis value for wavelength λ and N is the number of parameters.

23. The computer implemented method of claim 21, wherein said estimated virtual biological parameter vector Pₑ comprises:
Pₑ=[ψ^{T}(λ)ψ(λ)]⁻¹ψ^{T}(λ)Rₑ(λ)
where ψ(λ) is a matrix with column-wise basis vectors with each element representing a basis value for wavelength λ, and T is a transpose operation.

24. The computer implemented method of claim 21, wherein said measured virtual biological parameter vector Pₘ comprises:
Pₘ=[ψ^{T}(λ)ψ(λ)]⁻¹ψ^{T}(λ)Rₘ(λ)
where ψ(λ) is a matrix with column-wise basis vectors with each element representing a basis value for wavelength λ, and T is a transpose operation.

25. The computer implemented method of claim 21, wherein said biophysics model comprises a model of multi-layered skin tissue, said biological parameter vector comprising any combination of: epidermal thickness, melanin concentration, dermal blood volume fraction, skin oxygen saturation, and a light scattering parameter.
